(19) United States
(12) Patent Application Publication
(10) Pub. No.: US 2017/0042272 A1
(43) Pub. Date: Feb. 16, 2017

(54) IMPACT-ABSORBING HEADGEAR LINER AND SKULL CAP

(71) Applicant: BOB FERGUSON, ELOY, AZ (US)

(72) Inventor: BOB FERGUSON, ELOY, AZ (US)

(21) Appl. No.: 15/336,605

(22) Filed: Oct. 27, 2016

Related U.S. Application Data
(63) Continuation-in-part of application No. 13/523,556, filed on Jun. 14, 2012, now abandoned.
(60) Provisional application No. 61/530,275, filed on Sep. 1, 2011.
(60) Provisional application No. 61/520,778, filed on Jun. 14, 2011.

(51) Int. Cl. A42B 3/12; A42B 3/28; A61B 5/00; A42B 3/08; A41D 13/015; A42B 3/04; A63B 71/08

(52) U.S. Cl. CPC A42B 3/121; A42B 3/127; A42B 3/046; A42B 3/283; A63B 71/081; A42B 3/08; A41D 13/015; A61B 5/6803; A61B 2562/0219

(57) ABSTRACT

A protective impact-absorbing headgear liner and impact sensing system for use with various types of helmets and protective gear or clothing. The lining material has unique impact absorbing properties to additionally protect a wearer from impact related injuries. The headgear liner has a band and crown which are variously shaped and positioned to received impact-absorbing pads. The position of the pockets depends on the helmet style. In one example the liner is a stretchable material. Impact absorbing pads as described herein may be used in a variety of clothing and protective gear to protect from impact injury. Examples are football shoulder ads, thigh pads, bicycle helmets and the like. The liner may also be an expanded foam. A preferred pad material is a gel containing a thermoplastic elastomer. The impact sensing system utilizes an impact sensor assembly to sense the force of impact received and transmit the data to a personal electronics device running an application program to process and display sensor data. Other data such as temperature or the like provided by other ancillary sensors may also be processed by the application program.

### CROSS-REFERENCE TO RELATED APPLICATIONS

This is a continuation-in-part of application Ser. No. 13/523,556, filed Jun. 14, 2012, the subject matter of which is incorporated herein by reference. application Ser. No. 13/523,556 claims the benefit of U.S. Provisional Patent Application No. 61/520,778 filed Jun. 14, 2011, and U.S. Provisional Patent Application No. 61/530,275 filed Sep. 1, 2011 the contents of which are hereby incorporated by reference.

### TECHNICAL FIELD

This description relates generally to protective, impact-absorbing headgear/helmet liner and more specifically, to a protective liner to be worn under various types of helmets for additional impact protection that includes a system to measure impact.

### BACKGROUND

Impacts resulting in head injuries are a common occurrence in many activities such as football, hockey, cycling and other activities. The problem is particularly critical in sports such as football as played at all levels. A recent report indicated that even at the NFL level, many players wear helmets that received low ratings for reducing the risk of concussion.

The term “concussion” is derived from the Latin term “concutere” and often results from head-on, helmet-to-helmet impacts. Some research also shows that head trauma is often caused by impact or blows to the side of the face as well as a result of direct head-on collisions.

Concussions may be associated or result in a variety of symptoms after an injury which vary widely in nature and seriousness with headache being the most common. Dizziness, vomiting, lack of motor coordination may follow. In addition to these, physical symptoms, cognitive symptoms include confusion and disorientation. Concussion-causing impacts may detrimentally affect various parts of the brain including the midbrain, the fornix, temporal and frontal lobes.

Individuals such as athletes subjected to repeated concussions may exhibit a decline in physical and mental performance over time. The cumulative effects may increase the chance of early development of Alzheimer's, brain swelling, neurological problems and even result in mortality. Often the amount impact is unknown. Impact measurement may be provided in a standard helmet in an effort to quantify impact to a helmet, however impact adjacent to the players skull with a material disposed between the helmet and the player's head has not been provided. Location of a sensor system closer to the player's head would tend to be more useful as it would measure impact closer to the skull, where it is felt.

As a result of medical concerns in the field of sports, various approaches have been undertaken in head protection which attempt to lessen or absorb the effect and severity of impacts and blows to the head, although, as pointed out, many athletes continue to wear helmets that do not perform well.

While these various approaches have, to some extent, improved helmet performance and measured impact, there nevertheless exists a need for further improvements in the field of headgear safety, and in particular to utilizing materials that more effectively absorb impact that are light weight, compact and reusable and also measure impact transmitted to a players head.

### SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

The present examples provide a separate, impact-absorbing helmet liner to be worn beneath various types of helmets. In one example, the liner is a skull-type cap of a stretchable, breathable material which will fit snugly on the head of the wearer. A circumferential band, preferably having an elasticized section, encircles the wearer's head, extending around the temporal, frontal and occipital areas. The circumferential band defines a plurality of pockets which may be variously sized and positioned to received impact-absorbing pads. The placement of the pockets may be selected in accordance with the type of helmet and activity, as the areas most susceptible to impact blows will vary with the activity. Generally the pockets are spaced circumferentially around the band and contain an impact-absorbing material which may be permanent inserts or in the form of removable and replaceable pads.

The liner also has a integral crown portion attached to the circumferential band that extends across the top of the wearer's head. The crown portion of the liner also defines at least one of a plurality of selectively positioned pockets which receive either permanent or removable pads. The crown pocket typically extend across the top of the head from the forehead area to the back of the neck. The pockets are typically constructed so that no fasteners are needed. But if desired pockets having closures such as loop-and-hook fasteners or the like may be constructed.

The impact or alternatively energy absorbing gel pads are preferably a gel material such as disclosed in U.S. Pat. No. 7,041,719 entitled “Shock Absorbing Compound” and U.S. Pat. No. 6,896,065. This particular impact absorbing gel material may be composed of an epoxidized vegetable oil, a thermoplastic polymer and a prepolymer. The energy absorbing compound is a polymeric compound which exhibits good energy-attenuating properties and is capable of absorbing repeat shocks without structural damage. The pads may be of varying thickness, depending on their placement and type of activity with which they are to be used. The use of this particular material tends to reduce the thickness of the pads needed, as its energy absorbing characteristics per unit volume tend to outperform those of other commonly utilized materials.

In an alternate embodiment, the liner may be an expanded foam backed with the impact absorbing gel material. Pockets are also provided for receiving impact-absorbing pads to absorb impacts to the head transmitted through a helmet or other equivalent heads gear. The pads are preferably removable.

Also included in an example of impact absorbing head gear liners including at least one integral impact sensor. The at least one sensor may include a transmitter and or receiver coupled wirelessly to an external device such as a smartphone, tablet computer, computer or the like. An application program is provided to collect and process sensor information and provide it to the remote device. The remote device may process the sensor data in order to determine measurements of impact received and the like. Many of the attendant features will be more readily appreciated as the same becomes better understood by reference to the following detailed description considered in connection with the accompanying drawings.

### DESCRIPTION OF THE DRAWINGS

The present description will be better understood from the following detailed description read in light of the accompanying drawings, wherein:

FIG. 1 is a perspective view of an example of a helmet liner according to the present invention including impact or energy absorbing gel pads;

FIG. 2 is a side view showing the helmet liner of FIG. 1 worn by a player for under helmet protection;

FIG. 3 is a bottom view of the helmet liner showing an illustrative pocket opening for accepting a gel pad;

FIG. 4 shows a front and top view of the helmet liner.

FIG. 5 is a perspective view showing the position of the gel inserts without the supporting fabric of the cap, and as they would be held in place by the fabric.

FIG. 6 shows a wearer wearing a football helmet showing the liner of the present invention in dotted lines.

FIG. 7 shows a top view of an impact absorbing helmet liner with an impact sensor.

FIG. 8 shows a pictorial view of the liner with a sensor located therein

FIG. 9 shows a front view of the impact absorbing headgear liner with an impact sensor

FIG. 10 shows a side view of the impact absorbing headgear liner with an impact sensor.

FIG. 11 shows a wireless system for coupling the sensor in the impact absorbing headgear liner to a remote electronics device.

FIG. 12 shows a block diagram of a remote electronics device including an applications program for capturing sensor data.

Like reference numerals are used to designate like parts in the accompanying drawings.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

The present examples uniquely provide a gel helmet liner that may be configured in any variety of configurations in order to utilize gel material as described herein, either alone or in combination with other materials to absorb shock applied to the helmet. The impact absorbing gel is soft enough to conform its shape to a certain extent that is useful in improving the fit of various items. The impact absorbing gel is particularly useful in that when used in helmets it has the ability to fill voids between the wearer and the helmet, making for a better fit, and better transfer of impact to the gel where the force of impact may be absorbed.

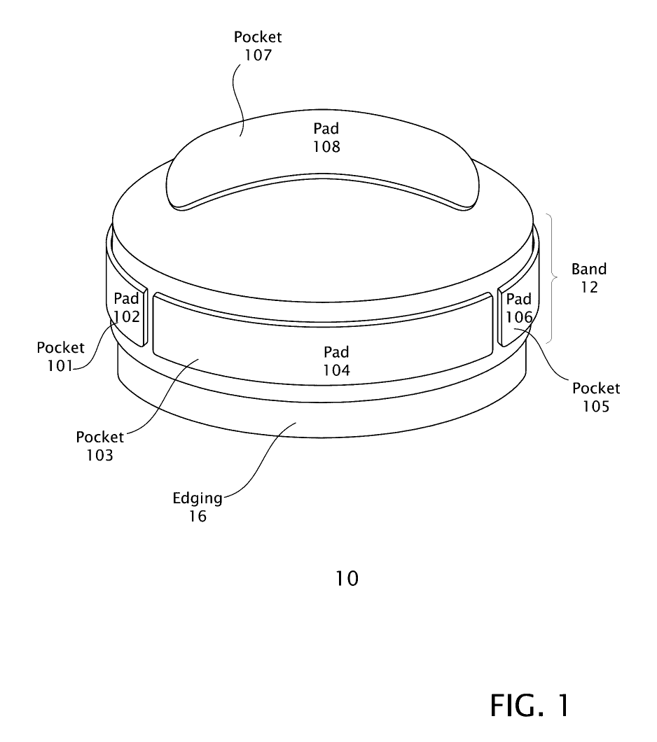

FIG. 1 is a perspective view of an example of a skull cap or helmet liner 10. The skullcap 10 includes a support liner 109 for a plurality of unique impact absorbing gel pads 102, 104, 106, 108. Such a liner may advantageously be worn under a hard helmet to distribute the force of impacts received in play or other activities. This liner shape also lends itself for use with certain styles and types of helmets which do not enclose as much of the wearer's head as do full face football, hockey goalie and similar helmets. In addition to football helmets, representative applications would be bicycle helmets, skiing protective headgear, skateboarding helmets and climbing helmets. The skull cap may be worn as shown, but may be reversed if desired. The helmet liner 10 includes a plurality of impact or energy absorbing gel pads such as those shown in this view 102, 104, 106, 108 which may be disposed in various pockets 101, 103, 105, 107 disposed at advantageous positions in a supporting cap 109.

The skull cap is unique over caps and helmets having protective liners. In the present invention the structure of the cap and the materials in the skull cap hold the impact absorbing materials firmly in place against a wearer's head to consistently protect a defined area of the head. A simple cap or helmet tends to shift position while worn, and because of such slippage may not consistently protect a desired portion of the head.

The supporting cap structure 109 that supports the pads shown 102, 104, 106, 108, and holds them in place, shown here is assembled from several components including a circumferential head-encircling band 12, and an integrally formed crown 14, and an edging 16. Alternatively each of these components may include sub-components as desired, for example additional pieces of material that may be used to form pockets 101, 103, 105, 107, as desired. These components may be made of the same or differing materials, fabric, mesh or the like as desired. In particular material that may be stretched, or that includes elastic may advantageously employed so that the cap stays in position on a player's head. The band 12 and crown 14 are both of a lightweight, breathable material such as a micro fiber, Spandex, etc. that is typically able to stretch in two directions. The various components may usually be assembled by sewing. Elasticized thread or non-elasticized thread may be used to sew the carious components as desired. Other equivalent methods of joining the component pieces may also be employed if desired.

In addition to the use of mesh material or netting perforations or vents (not shown) may also be provided at selected areas in the crown and band for ventilation and to allow heat to escape from the wearer's head. Perforations may also be provided in the crown. In addition pads having apertures disposed therein may be used to increase comfort.

The supporting cap structure 109 may be provided in various sizes for use by both adult and younger athletes, although stretchability will adapt for use by several head sizes. The band 12 has a series of generally rectangular pockets101, 103, 105 in which shock absorbing gel pads 102, 104, 106 may be disposed extending about the band. The pockets pockets101, 103, 105, each have a slot or opening (not shown) through which impact-absorbing pads may be inserted and removed. The slots may include optional fasteners if desired.

The selected pads 102, 104, 106, 108 will generally conform to the shape of the associated pocket 101, 103, 105, 107 and are shown as removable, generally rectangular inserts. Generally elongated, rectangular pad configurations for insertion in the band may be provided however other alternative shaped pads may be provided, including additional pads fit in the crown 14 in similarly configured pockets in this particular example. The shapes described above could be selected for use in football helmets or the like. The pads 102, 104, 106, 108 may be a foam or similar material but are preferably gel pads as described below.

While various types of impact absorbing materials may be used for pads such as foam and the like. However, a unique impact absorbing gel material has been found to be very effective in absorbing and distributing impact. The gel pads of this material may be cut formed or otherwise shaped from stock having a variety of thicknesses including 2 mm, 8 mm, 12 mm and 14 mm exemplary thicknesses. The material is also advantageously compact given its impact absorbing qualities, typically with pads of about 25% of the thickness of other padding materials, equivalent or better impact absorbing characteristics are obtained. A preferred gel material is a non-toxic material with high energy absorbing capacity. One such preferred material is an epoxidized vegetable oil containing a thermoplastic elastomer along with a prepolymer.

The impact or energy absorbing gel which is further described in U.S. Pat. No. 7,041,719 by Kriesel and Goodenough, for “Shock Absorbing Compound” filed Sep. 26, 2003; U.S. Pat. No. 6,896,065 by Kriesel and Lyle for “Hoofed Animal Pad” filed Jul. 3, 2003; and U.S. Pat. No. 6,588,511 by Kriesel and Lyle for “Hoofed Animal Pad” filed Mar. 7, 2002, the disclosures of which is hereby incorporated by reference.

The gel material can be provided with stabilizers to resist fungus and bacteria, an important characteristic in athletic wear which is subject to heat, moisture and perspiration. In addition the gel material may be perforated with apertures of any convenient shape or shapes. Perforation allows for air circulation and improved cooling so that perspiration may evaporate more readily, increasing comfort to the wearer.

The gel material may also be combined with other substances, either in a mixture, layered structure or other suitable combination to form a pad. Typical structures might include gel disposed on a layer or layers over a layer or layers of styrene foam, an air bladder, or a mixture of gel material with air disposed throughout the gel. A particular example of pad material may be a mixture of styrene plastic and rubber blended to form an impact resistant substance.

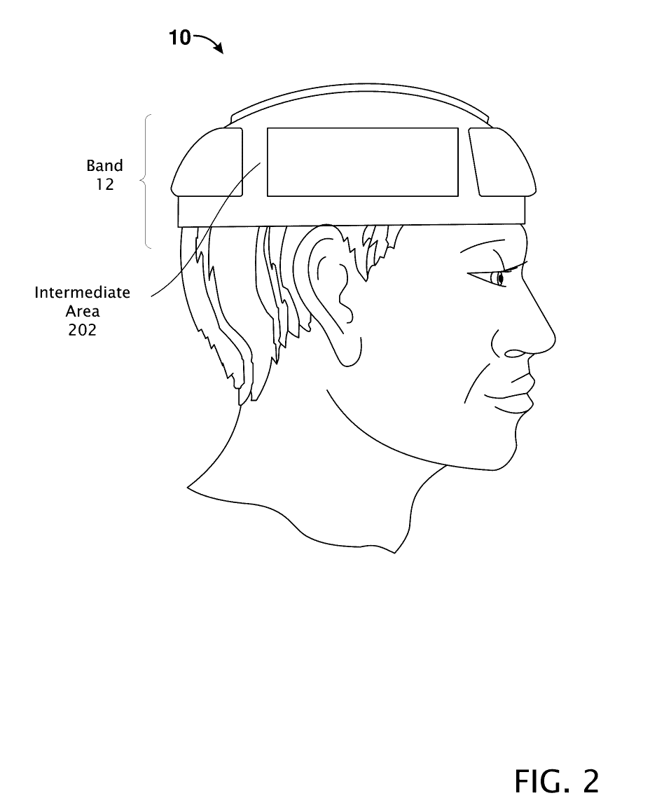

FIG. 2 is a side view showing the helmet liner of FIG. 1 worn by a player for under helmet protection prior to a helmet being put on. The material is stretchable so the liner will snugly and firmly encircle at least the top portion of the wearer's head. When worn, the band 12 extends around the frontal, temporal an occipital region of the head and the crown.

In alternative example one or more elastic sections positioned intermediate the impact-absorbing pad receiving pockets 202 may be provided to enhance stretchability, fit and adaptability of the liner to various head sizes. The crown may also be provided with a plurality of pockets to receive impact-absorbing pads, either permanent or replaceable to protect the head as needed in other desired applications.

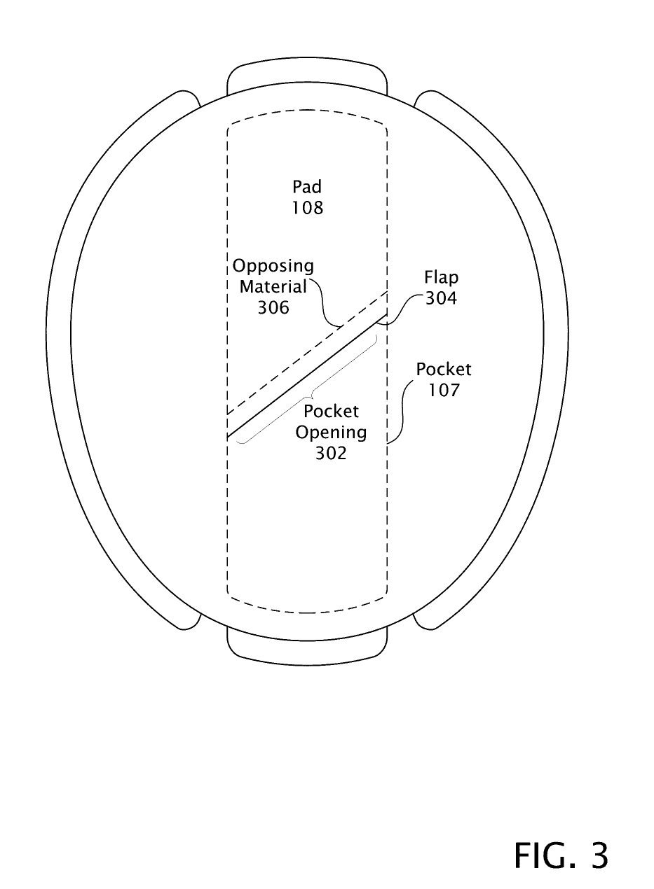

FIG. 3 is a bottom view of the helmet liner showing an illustrative pocket opening 302, disposed in a pocket 107 for accepting a gel pad 108. Each pocket disposed in the helmet liner 10 may have similar openings for removal or insertion of the pads. As seen a diagonal 304 flap is formed that tends to overlap the abutting fabric similarly cut, so that when a pad 108 is inserted the gel material is not exposed, as it might prove uncomfortable to the wearer.

Gel pads 108 may be easily disposed in a pocket enclosure of this type as they are flexible. In this example a flap 304 of material overlaps an opposing piece of material 306 to form an opening 302 in which a pad 108, may easily be inserted or removed without the need for hardware, which might be unsafe or uncomfortable for a wearer. The material is flexible allowing the pad to be positioned. In use the material is taught on the wearer's head closing the cavity with the taught material, and keeping the pad 40 in place. Providing removable pads is useful as the pads may be removed and the skull cap may be washed separately.

The opening may optionally include cooperable loop-and-hook fasteners to allow the convenient and selective insertion and removal of pads 108, although a slot or aperture on the interior of the crown may be sufficient to retain the pads in most cases.

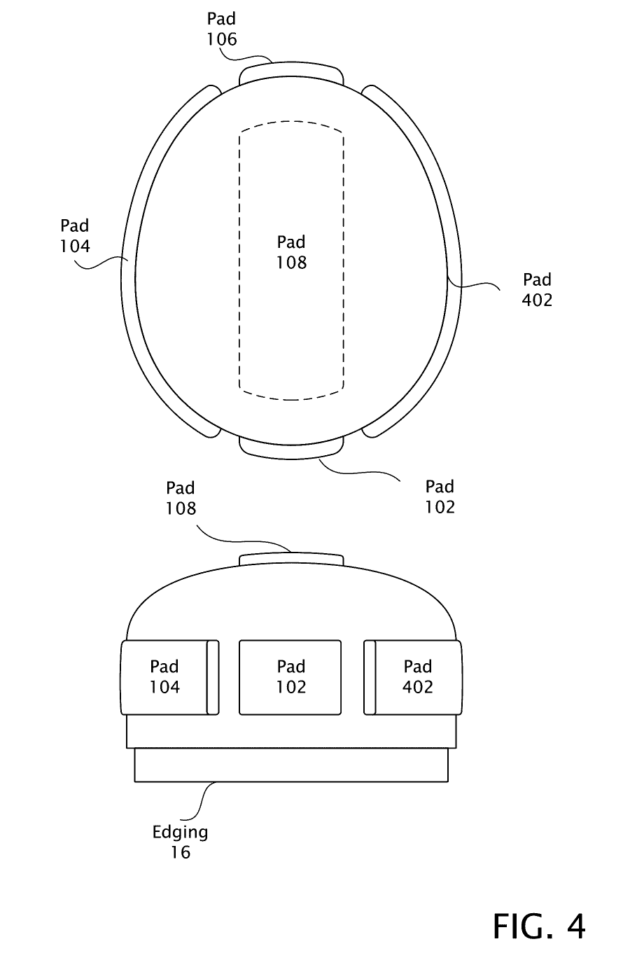

FIG. 4 shows a front and top view of the helmet liner. In this view all of the pad locations of the five gel pads 102, 104, 106, 108, 402 of this example are shown. In an alternative example of the helmet liner, a chin strap (not shown) may be provided. A chin strap serves to keep the liner in place-especially when the liner may be worn without a helmet such as during flag football games.

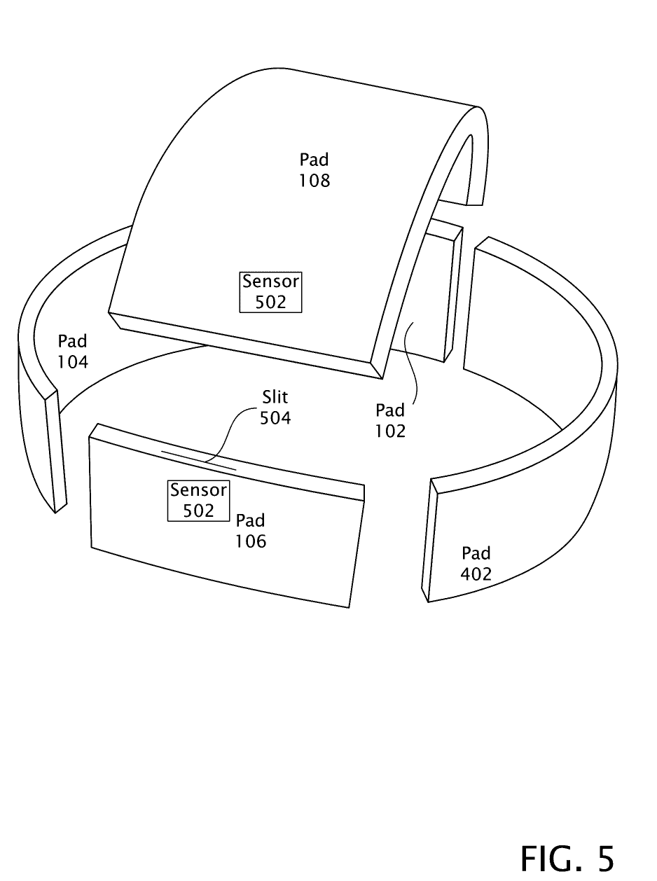

FIG. 5 is a perspective view showing the position of the gel inserts 102, 104, 106, 108, 402 without the supporting fabric of the cap, and as they would be held in place by the fabric. As can be see the inserts are quite thin, and flexible, so that they may be cut from a planar sheet but still remain flexible enough to conform to the shape of the wearer's head.

In addition one or more pieces 102, 106 may be used to adjust the liner size. Impact absorbing gel pieces may act as a self-adjusting gel tail fitting piece 102 and forehead fitting piece 106, here shown as a rectangles or alternatively as squares. In alternative examples the fitting piece may be other shapes such as round oval, having rounded corners and the like. The fitting piece or pieces allows size adjustment by selecting a suitably sized fitting piece allowing a single sized side or crown pieces to be used. Alternatively fewer side and crown piece variations in size might be needed. The fitting piece additionally provides additional protection over leaving a gap to allow for sizing.

Additionally impact sensors, or impact sensor assemblies 502 may be disposed at various places in the pads 102, 104, 106, 108. In this example a slot 504 facilitates mounting of the sensor by inserting the sensor into the slit into a pocket disposed internal to particular pad holding the sensor.

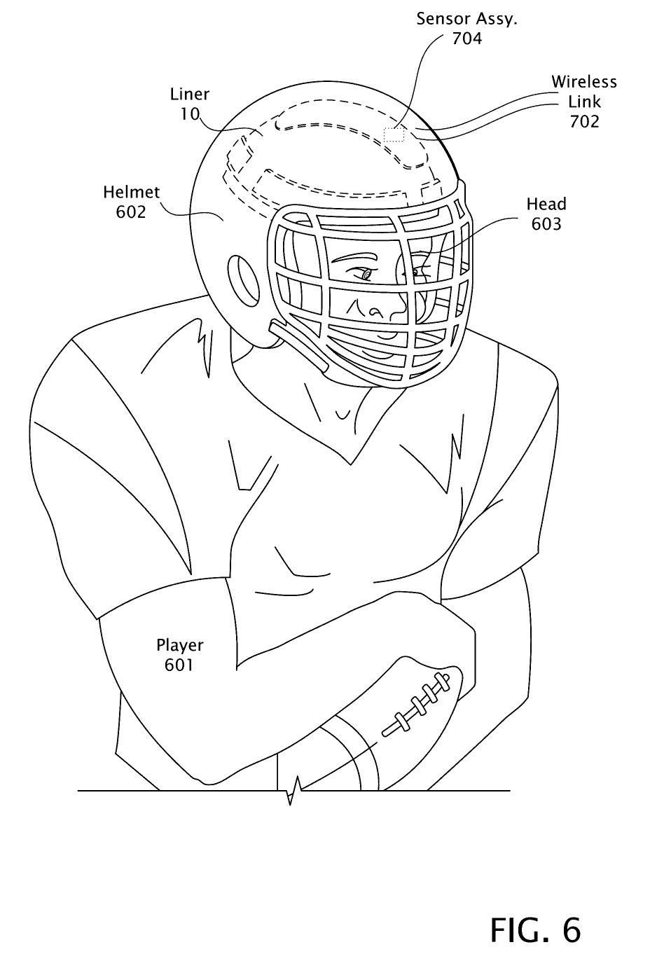

FIG. 6 shows a player 601 wearing a football helmet 602 with the liner 10 between the player's head 603 and his conventional football helmet 602 showing the liner 10 of the present invention in dotted lines. As shown the addition of the impact-absorbing liner or skull cap 10 provides a shock absorbing under layer to typical athletic head gear 602. In order to accommodate the impact-absorbing liner or skull cap a conventional helmet of a size can be selected. In an alternate example the impact-absorbing liner or skull cap may be used in conjunction with a simple hard shell helmet, without padding, or having any padding removed. In a further alternative example the impact-absorbing liner or skull cap may be provided with hardware such as hook and loop tape, or the like to prevent its shifting within the helmet.

There are various shapes and configurations for the impact-absorbing gel pad(s). The various embodiments of the liner of the present invention can be adapted to the requirements of the individual and the activity in which the wearer participates.

A liner may be constructed that includes mandible extensions which extend downwardly towards the chin, and may include a chin strap to help secure the cap in place. Alternatively the mandible extensions may be omitted, with a chin strap still provided. The mandible extensions 350 have openings 375 for better sound transmission. The openings may also receive impact-absorbing inserts 340 insertable in or around the ear openings.

And in further alternative examples the impact-absorbing liner or skull cap may be disposed as an integral part of the helmet. In such a configurations either the liner its self may be made removable for washing, the pads alone may be removable for washing, or both may be removable for washing.

In alternative examples tubes or strips of the previously described gel may be disposed in a skull cap. This example in particular allows roughly pencil sized plastic (or its equivalent) of gel to be inserted in pockets or cavities disposed in a skull cap for shock absorption. The strips or tubes may radiate from the crown, may form concentric circles, a spiral or the like. Weight and ventilation tend to be improved in these examples.

In yet another alternative examples of the skullcap a “T” shaped gel pad or a circular gel pad in cooperation with rectangular pads in inserted into a support liner having suitably formed pockets. These examples advantageously utilize the sewn envelopes previously described.

In yet another alternative example of the skull cap size adjustment can be achieved without elastic, or in addition to it. Fabric tails may be coupled to the skull cap by sewing or other suitable bonding processes. The tails may be tied together to adjust the fit of the skull cap. Tying would typically be done in the back, but tying in the front or other convenient locations would be possible. Also the skull cap could be fabricated as a scarf with pockets which is tied up and worn so that the position of the impact absorbing gel pads provides the desired protection under the headgear.

Finally the impact absorbing gel pads utilized and described herein may be used in athletic gear and protective gear as either additional protection disposed between the conventional protective gear and the wearer, or incorporated into the design of such protective gear. The application of the gel material to athletic equipment and the like for impact reduction is wide ranging. The impact absorbing gel pads may also be sewn into articles of clothing, such as hip pads, knee pads and the like. Also, the gel may be incorporated into mouth guards, for example by providing an insert into the tray of a mouthpiece so that the teeth close upon the impact absorbing gel protecting them from damage.

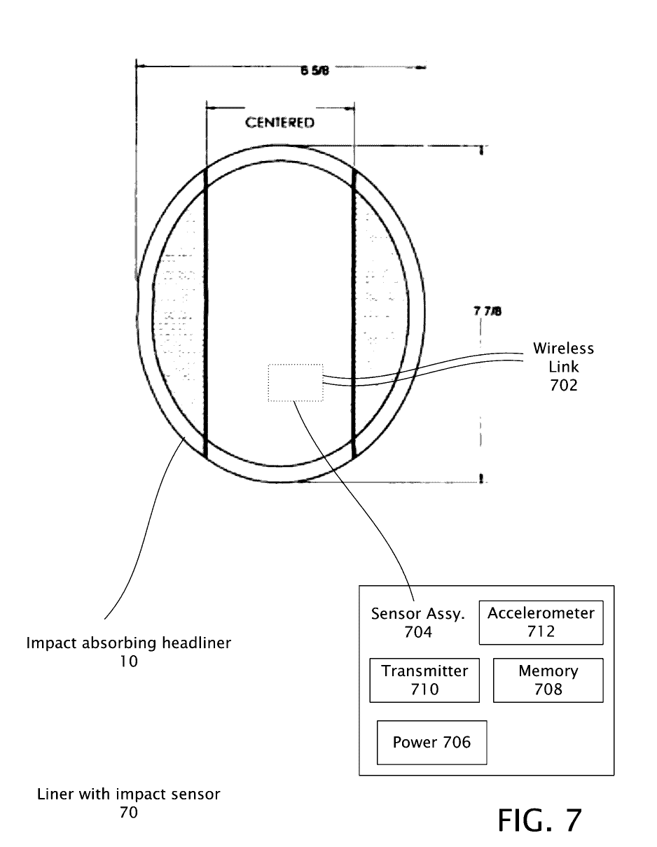

FIG. 7 shows a top view of an impact absorbing helmet liner 10 constructed as previously described herein, with an impact sensor electronics 704. The sensor 704 is communicatively coupled via wireless link 702 to remote electronics (not shown). Adding an impact sensing system may help record and monitor impacts when they occur. And since the sensor is mounted into the impact absorbing helmet liner impact to the skull is more accurately determined. The sensor may be sewn into the liner 10, disposed in a pocket, or otherwise suitably be coupled to the liner 10. The sensor may be placed under or in gel in the front, top or back of gel cap. Another option is to make a separate pocket for the sensor so it can be easily removed for washing. The sensor may also be covered in a plastic film to protect from moisture. The sensor can also record impact of falls which could be reviewed by Physician to determine severity of fall. Also, temperature may be monitored by a physician to warn of impending heat exhaustion, heat stroke, or the like.

The sensor assembly 704 is typically, 2 to 3 mm thick ¾ in.× ¾ in. in dimension, although these dimensions are not limiting and but exemplary. The sensor assembly includes a power source 706, such as a battery, and has a battery life comparable to that of a watch. The sensor may be identified by transmission of a serial number disposed in an integral memory 708 (hard wired or programmable) to a remote monitor via a wireless link. A suitable transmitter 710 included, converts data from the accelerometer into a wireless signal (typically RF) for transmission to a remote electronic device (not shown). In an alternative example a receiver may be included to facilitate calibration of the sensor, or other programming of the unit that may be desired.

Accordingly the system may monitor one, or a plurality of sensors 712 (in other player's helmets- or in the same helmet). The sensor may record longitudinal, lateral, vertical and resultant acceleration along with temperature and other desirable metrics. Accordingly, the sensor may be of the type known as an accelerometer.

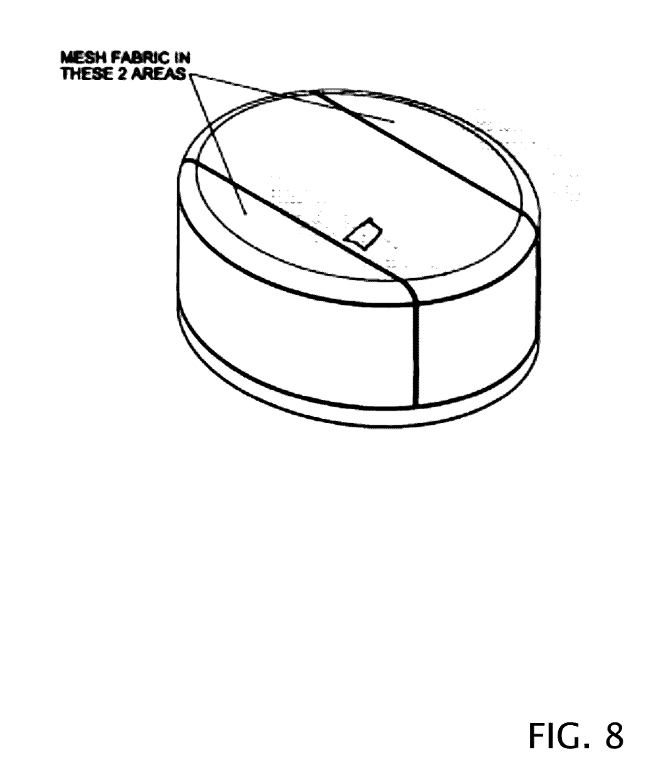

FIG. 8 shows a pictorial view of the liner with a sensor located therein. Here the sensor 704 is on the top of the cap 10, disposed under the gel material between the players head and the gel. A pocket or aperture in the gel tends to keep the sensor in a stable position. In alternative examples one or more sensors may be disposed anywhere that is desired in the cap, in the fabric, or the gel material.

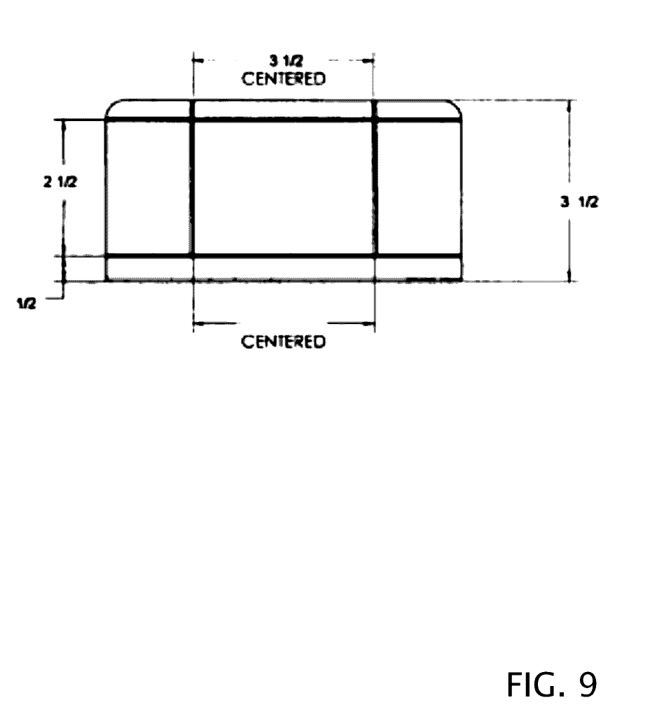

FIG. 9 shows a front view of the impact absorbing headgear liner with an impact sensor, with exemplary dimensions.

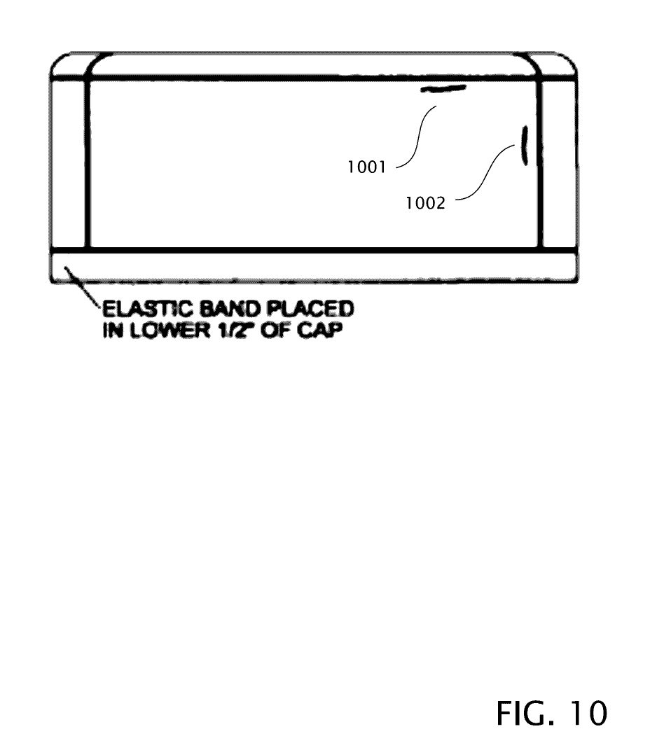

FIG. 10 shows a side view of the impact absorbing headgear liner with alternative impact sensor assembly locations 1001, 1002.

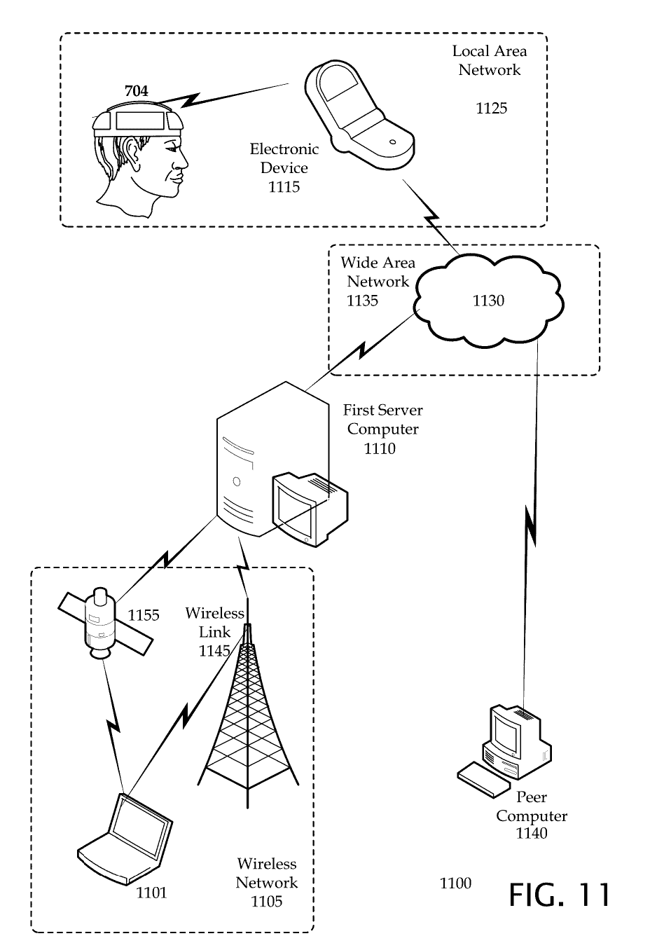

FIG. 11 shows a wireless system for coupling the sensor in the impact absorbing headgear liner to a remote electronics device. The sensor can be used with or without a transmitter. The transmitter may relay data to an electronic device such as an I phone, Android, Ipad, lap top, or the like. The sensor sends data to phone or Android to be reviewed either electronically or manually. Data may be stored in the sensor assembly for later or periodic download, or may be monitored continuously.

FIG. 11 is an exemplary network 1100 in which the system for sensing impact may be implemented. Through computer networking the sensor data may be sent to many different locations via networked connections, such as the wireless device 1115, or remote computers 1101, 1140. Device 1115 may couple to a user's computer 1101 1140 through various conventionally constructed networks 1125, 1135, 1145, 1155, 1140.

In the local area network the users computer is typically part of the local area network 1125 which may include a plurality conventional computers (not shown) and conventional peripheral equipment (not shown) coupled together utilizing topologies (token, star and the like) and switching equipment known to those skilled in the art. Those skilled in the art will realize that other processor equipped devices such as televisions and VCRs with electronic program guides, cellular telephones, appliances and the like may be coupled to the internet utilizing conventional techniques known to those skilled in the art.

A typical local area network 1125 may include a conventionally constructed ISP network in which a number or plurality of subscribers utilize telephone dial up, ISDN, DSL, cellular telephone, cable modem, or the like connections to couple their computer to one or more server computers 1115 that provide a connection to the world wide web 1135 via the internet 1130.

Wide area network or world wide web 1135 is conventionally constructed and may include the internet 1130 or equivalent coupling methods for providing a wide area network. As shown a conventionally constructed first server computer 1110 is coupled to conventionally constructed second server computer 1115 through a conventionally constructed internet connection to the world wide web 1130.

In a peer to peer network a Peer computer 1140 is conventionally constructed to couple to the internet 1130 utilizing peer to peer network technology. Peer computer 1140 may couple to a plurality of similarly connected peer computers in a peer to peer network (not shown), or to other computers 1101, 1120 that are part of conventionally constructed networks 1125, 1135.

In a conventional wireless network 1105 sensor in the skull cap 704 may be coupled to the internet 1130 via a conventionally constructed wireless link 1145. The wireless link may include cellular, and satellite technology 1155 to provide the link. Such a wireless network may include a conventionally constructed first server computer 1110 through an electronic device 1115 such as a PDA, tablet computer, laptop, desktop, smartphone, or the like, typically provided to collect data or manage connections to a wide area network such as the internet.

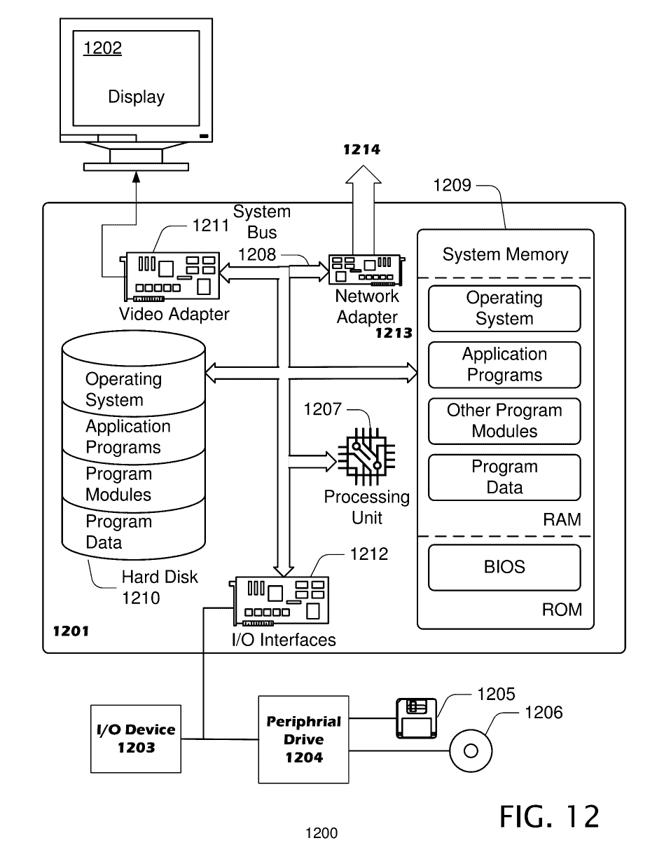

FIG. 12 shows a block diagram of a remote electronics device including an applications program for capturing sensor data. The exemplary computing environment 1200 in which the impact sensing system described in this application, may be implemented is shown. Exemplary computing environment 1200 is only one example of a computing system and is not intended to limit the examples described in this application to this particular computing environment.

For example the computing environment 1200 can be implemented with numerous other general purpose or special purpose computing system configurations. Examples of well known computing systems, may include, but are not limited to, smart phones (android, apple, windows and the like), tablet computers, personal computers, hand-held or laptop devices, microprocessor-based systems, multiprocessor systems, consumer electronics, cellular telephones, PDAs, and the like.

The computer 1200 includes a general-purpose computing system in the form of a computing device 1201. The components of computing device 1201 can include one or more processors (including CPUs, GPUs, microprocessors and the like) 1207, a system memory 1209, and a system bus 1208 that couples the various system components. Processor 1207 processes various computer executable instructions, including those to ** and to control the operation of computing device 1201 and to communicate with other electronic and computing devices (not shown). The system bus 1208 represents any number of several types of bus structures, including a memory bus or memory controller, a peripheral bus, an accelerated graphics port, and a processor or local bus using any of a variety of bus architectures.

The system memory 1209 includes computer-readable media in the form of volatile memory, such as random access memory (RAM), and/or non-volatile memory, such as read only memory (ROM). A basic input/output system (BIOS) is stored in ROM. RAM typically contains data and/or program modules that are immediately accessible to and/or presently operated on by one or more of the processors 1207.

Mass storage devices 1204 may be coupled to the computing device 1201 or incorporated into the computing device by coupling to the buss. Such mass storage devices 1204 may include a magnetic disk drive which reads from and writes to a removable, non volatile magnetic disk (e.g., a “floppy disk”) 1205, or an optical disk drive that reads from and/or writes to a removable, non-volatile optical disk such as a CD ROM or the like 1206. Computer readable media 1205, 1206 typically embody computer readable instructions, data structures, program modules and the like supplied on floppy disks, CDs, portable memory sticks and the like. In this instance program instructions to read impact data from the sensor is provided. In addition the raw sensor data may be analyzed and processed for display to the user through a UI or equivalent interface. In addition data from multiple sensors and or multiple players may be sorted and managed by an application conventionally constructed to collect and manipulate the data.

Any number of program modules can be stored on the hard disk 1210, Mass storage device 1204, ROM and/or RAM 1209, including by way of example, an operating system, one or more application programs, other program modules, and program data. Each of such operating system, application programs, other program modules and program data (or some combination thereof) may include an embodiment of the systems and methods described herein.

A display device 1202 can be connected to the system bus 1208 via an interface, such as a video adapter 1211. A user can interface with computing device 702 via any number of different input devices 1203 such as a keyboard, pointing device, joystick, game pad, serial port, and/or the like. These and other input devices are connected to the processors 1207 via input/output interfaces 1212 that are coupled to the system bus 1208, but may be connected by other interface and bus structures, such as a parallel port, game port, and/or a universal serial bus (USB).

Computing device 1200 can operate in a networked environment using connections to one or more remote computers through one or more local area networks (LANs), wide area networks (WANs) and the like. The computing device 1201 is connected to a network 1214 via a network adapter 1213 or alternatively by a modem, DSL, ISDN interface or the like.

It will be obvious to those skilled in the art to make various changes, alterations and modifications to the invention described herein. To the extent such changes, alterations and modifications do not depart from the spirit and scope of the appended claims, they are intended to be encompassed therein.

Those skilled in the art will realize that the process sequences described above may be equivalently performed in any order to achieve a desired result. Also, sub-processes may typically be omitted as desired without taking away from the overall functionality of the processes described above.

1. An elasticized under helmet skull-cap comprising:
a circular head-encircling band having a crown;
the band and crown each defining at least one pocket;
an impact-absorbing gel pad in the pocket; and
an electronic sensor disposed in the impact absorbing gel pad to measure impact and whereby an elasticized material in the under helmet skull-cap holds the scull-cap in place on a wearer's head and holds the impact absorbing gel pad in position against the wearer's head.

2. The liner of claim 1 wherein the pads are removable from the pockets.

3. The liner of claim 2 wherein the pockets are formed by overlapping pieces of fabric.

4. The liner of claim 1 wherein the pads are an impact-absorbing gel.

5. The liner of claim 4 wherein the gel is a thermoplastic resin.

6. The liner of claim 1 wherein the band and crown are an elasticized, stretchable material.

7. The liner of claim 1 wherein the impact absorbing gel pad includes an expanded foam disposed on the impact absorbing gel pad.

8. The liner of claim 1 wherein the liner has a tail piece formed from impact absorbing gel to adjust a size of the liner.

9. The liner of claim 1 wherein the location of the pockets are selected to provide impact-resistance in areas based on the type of helmet and activity.

10. The liner of claim 1 in which the sensor includes an accelerometer, a power source, and a wireless transmitter configured such that accelerometer data may be transmitted wirelessly.

11. The liner of claim 1 further comprising an application program disposed on a personal electronic device for receiving information from the sensor wirelessly.

12. A protective gear system comprising:
an article of protective gear to designed to protect a wearer from an impact related injury;
a thermoplastic elastomer material disposed between the article of protective gear and a wearer of the protective gear to further absorb an impacting force whereby the wearer is further protected; and
a sensor disposed in the thermoplastic elastometer to measure an impact to the user.

13. The protective gear system of claim 12 further comprising an application program operating on a personal electronic device to collect impact data wirelessly from the sensor.

14. The protective gear system of claim 13 in which force of impact is analyzed.

15. An under sports helmet liner comprising:
a circular head-encircling band having a crown made from a breathable material to allow the evaporation of sweat and dissipate heat;
said band and crown each defining at least one pocket, without fasteners, said pocket being stretchable said crown extending across the top of a wearer's head from a forehead area to a back of the neck;
at least one impact-absorbing re-usable gel pad of epoxidized vegetable oil containing a thermoplastic elastomer and a prepolymer and stabilizers to resist fungus and including apertures in the pad for ventilation, in said pocket, said pads are disposed to protect designated areas of a wearer's skull from impact transmitted through a commercially available helmet worn over the helmet liner by further absorbing the impact, and the commercially available helmet may be used due to reduced thickness of the re-usable gel pad having enhanced energy absorbing capabilities, whereby protection provided by the sports helmet is enhanced; and
an electronic impact sensor disposed in the least one impact-absorbing re-usable gel pad of epoxidized vegetable oil containing a thermoplastic elastomer and a prepolymer and stabilizers to resist fungus and including apertures in the pad for ventilation.

16. The liner of claim 15 wherein the pads are removable from the pockets.

17. The liner of claim 16 wherein the pockets are formed by overlapping pieces of fabric.

18. The liner of claim 15 wherein the pads are an impact-absorbing gel.

19. The liner of claim 18 wherein the gel is a thermoplastic polymer and a prepolymer.

20. The liner of claim 15 wherein the sensor is coupled to a portable electronic device through a wireless network.
